(19) United States
(12) Patent Application Publication
(10) Pub. No.: US 2020/0323170 A1
(43) Pub. Date: Oct. 15, 2020

(54) Solar-Powered Remote Monitoring Tag System for Animals

(71) Applicants: Derek S Heeger, (US); Roper Solutions, Inc., Las Cruces, NM (US)

(72) Inventors: Maeve Garigan, Las Cruces, NM (US); Derek S. Heeger, Albuquerque, NM (US)

(21) Appl. No.: 16/846,366

(22) Filed: Apr. 12, 2020

Related U.S. Application Data
(60) Provisional application No. 62/833,630, filed on Apr. 12, 2019.

(51) Int. Cl. A01K 11/00; H04Q 9/00; H04W 4/02; H04W 4/029; H04B 5/00; A01K 29/00; A61B 5/01; A61B 5/0205; A61B 5/00

(52) U.S. Cl. CPC A01K 11/004; H04Q 9/00; H04W 4/027; H04W 4/029; H04B 5/0068; A01K 29/005; A61B 5/02438; A61B 5/01; A61B 5/0205; A61B 5/6815; A61B 5/6839; H04Q 2209/886; H04Q 2209/40; A01K 11/008

(57) ABSTRACT

A solar-powered remote monitoring tag for beef cattle and other animals typically inhabiting outdoor environments combines animal health and activity monitoring with geolocation information. The tags communicate with a base station using radio communications, which in turn uses satellite internet services to communicate with a remote computing server. The remote computing server provides a user interface, such as a mobile app, that provides geospatially enabled information involving the health, activity, and location of monitored animals, thus providing timely and evidence-based decision support to the end user. The remote monitoring tags may also provide for close range communication using a near field communication transponder.

### TECHNICAL FIELD

The present invention relates to the field of animal management, and in particular to an apparatus for remote monitoring of the health, location and activity of individual animals in outdoor environments.

### BACKGROUND ART

There are approximately 54 million adult beef cattle in the United States and most of these animals are born and raised in outdoor environments such as pastures, rangelands and forests, making them vulnerable to predation, theft, injury, and disease. Cattle are inspected and managed manually, which is both labor-intensive and prone to mistakes. Operators of beef farms and ranches want to provide a healthy and safe environment for their cattle to live in, as this creates a productive and valuable herd. Advantageous practices such as grazing management and artificial insemination are particularly labor intensive and require specialized skills, making them impractical or infeasible for many producers to implement. While they want to maximize the performance of their herds, preserve grazing lands, and provide an abundant food supply, beef producers have had few tools to help them monitor and manage their cattle.

Traditionally, beef cattle have been monitored visually, with a common solution being visible ear tags that are attached through one of the animal's ears, with a flap having a visible identifier on it. The reliance on visual inspection creates significant inefficiencies in both animal care and herd management.

### SUMMARY OF INVENTION

One general aspect includes a solar-powered tag for an animal that primarily inhabits outdoor environments. The solar-powered tag also includes a housing, attachable to an animal; a solar panel, disposed within the housing and providing electrical power for the solar-powered tag; an accelerometer, disposed within the housing, that when operable measures movement of the animal; a geolocation sensor, disposed within the housing, that when operable determines a geolocation of the animal; a radio, disposed within the housing, having a stated range of at least 10 km; and a microcontroller, disposed within the housing and powered by the solar panel, connected to the accelerometer, the geolocation sensor, and the radio, and programmed to collect and analyze data received from the accelerometer and geolocation sensor and report the analyzed data and a timestamp information to a base station via the radio.

A second general aspect includes a solar-powered base station for use in an outdoor area. The solar-powered base station also includes a solar panel for providing electrical power to the solar-powered base station; a satellite internet terminal; a radio having a stated range of at least 10 km; and a microcontroller, powered by the solar panel, programmed to: communicate with a plurality of tags attached to monitored animals in the outdoor area using the radio, to collect analyzed sensor data and timestamps from the tags; communicate with a satellite internet service using the satellite internet terminal; and report the collected analyzed sensor data and timestamps via the satellite internet service to a remote computing server.

A third general aspect includes a system for monitoring animals in outdoor areas a plurality of solar-powered tags, each solar-powered tag may include: a housing, attachable to an animal; a solar panel, disposed within the housing and providing electrical power for the solar-powered tag; an accelerometer, disposed within the housing, that when operable measures a movement of the animal; a geolocation sensor, disposed within the housing, that when operable determines a geolocation of the animal; a radio, disposed within the housing, having a range of at least 3 miles; and a microcontroller, disposed within the housing and powered by the solar panel, connected to the accelerometer, the geolocation sensor, and the radio, and programmed to collect and analyze data received from the accelerometer and geolocation sensor and report the analyzed data and a timestamp via the radio. The system also includes a solar-powered base station, designed for placement in the outdoor area at a water source for the monitored animals, may include: a solar panel for providing electrical power to the solar-powered base station; a satellite internet terminal; a radio having a stated range of at least 10 km; and a microcontroller, powered by the solar panel, programmed to: communicate with the plurality of solar-powered tags using the radio to collect the analyzed data and the timestamps from the solar-powered tags; and communicate with a satellite internet service using the satellite internet terminal; and report the analyzed data and the timestamps to a remote computing server. The system also includes a remote computing server, programmed to communicate with the base station via the satellite internet service, where the remote computing server collects, aggregates, stores and analyzes information collected from the plurality of solar-powered tags.

A fourth general aspect includes a method for monitoring health. The method also includes attaching a solar-powered tag to a monitored animal; collecting and analyzing sensor data and assigning a timestamp corresponding to the monitored animal by the solar-powered tag, transmitting the analyzed sensor data and the timestamp to a solar-powered base station using a radio having a stated range of at least 10 km, receiving the analyzed sensor data and the timestamp from the monitored animal by the solar-powered base station, transmitting the analyzed sensor data and the timestamp corresponding to the monitored animal via satellite-based internet to a remote computing server, analyzing the sensor information and the timestamp received by the remote computing server, and providing geospatially enabled animal information to an end user via a user interface that communicates with the remote computing server.

### BRIEF DESCRIPTION OF DRAWINGS

The accompanying drawings, which are incorporated in and constitute a part of this specification, illustrate an implementation of apparatus and methods consistent with the present invention and, together with the detailed description, serve to explain advantages and principles consistent with the invention. In the drawings,

FIG. 1 is a block diagram illustrating a system for cattle monitoring according to one embodiment.

FIGS. 2A, 2B, 2C, and 2D are views of elements of various embodiments of a solar-powered remote monitoring tag and placement of the tag on a cow's ear.

FIG. 2E is a perspective view of an embodiment of a solar-powered remote monitoring tag for use with a collar or harness.

FIG. 3 is a flowchart illustrating an overview of the operation of solar-powered remote monitoring tags according to one embodiment.

FIG. 4 is a block diagram illustrating electronics used to implement a solar-powered remote monitoring tag according to one embodiment.

FIG. 5 is a block diagram illustrating components of a solar-powered base station for a remote animal monitoring system according to one embodiment.

### DESCRIPTION OF EMBODIMENTS

In the following description, for purposes of explanation, numerous specific details are set forth in order to provide a thorough understanding of the invention. It will be apparent, however, to one skilled in the art that the invention may be practiced without these specific details. In other instances, structure and devices are shown in block diagram form in order to avoid obscuring the invention. References to numbers without subscripts are understood to reference all instance of subscripts corresponding to the referenced number. Moreover, the language used in this disclosure has been principally selected for readability and instructional purposes, and may not have been selected to delineate or circumscribe the inventive subject matter, resort to the claims being necessary to determine such inventive subject matter. Reference in the specification to “one embodiment” or to “an embodiment” means that a particular feature, structure, or characteristic described in connection with the embodiments is included in at least one embodiment of the invention, and multiple references to “one embodiment” or “an embodiment” should not be understood as necessarily all referring to the same embodiment.

Although some of the following description is written in terms that relate to software or firmware, embodiments can implement the features and functionality described herein in software, firmware, or hardware as desired, including any combination of software, firmware, and hardware. References to daemons, drivers, engines, modules, or routines should not be considered as suggesting a limitation of the embodiment to any type of implementation.

In sum, reliance on visual inspection for the purposes of animal health and location monitoring is inefficient and causes significant losses and economic harm. The disclosure below directly addresses this problem by radically improving oversight of animal health and location through remote monitoring with embodiments that are compatible with end user workflows and management techniques. Remote monitoring of beef cattle is not a new idea; however, the supporting technologies required for a practical technological solution are recent. The technological solutions disclosed below for remote monitoring of beef cattle are not unique to cattle and can be widely applied to many types of animals that inhabit outdoor environments, including other types of domesticated animals and wild animals.

Below are several scenarios that illustrate how beef producers could use disclosed embodiments to improve management and radically reduce losses.

A steer is diseased as shown by his reduced feeding and rumination activity and movement patterns. The beef producer would receive an alert to their mobile device to notify them of the steers' health condition and geolocation. The beef producer could then treat or quarantine the sick steer before disease spreads throughout the herd.

A herd being harassed by a predator shows abnormal activity and the beef producer is alerted to the suspected predator and precise geolocation. This would allow the beef producer to immediately intervene, either in person or by dispatching a small drone to the geographic coordinates associated with the herd's location.

A cow is coming into standing heat as indicated by her movements and activities. The beef producer would be notified of her estrus status and location via their mobile device. A cow is at her most fertile 12-18 hours after she comes into standing heat, making that time window ideal for artificial insemination. The use of artificial insemination is shown to increase net farm cash income by 20-25% and would be made practical for many beef producers with remote estrus detection.

A cow having difficulty calving triggers an alert and the beef producer takes immediate action without having to guess the cow's health or her precise location. Nearly 13% of nonpredatory losses are due to calving-related problems.

A beef producer receives an alert indicating that several cattle have ranged outside a preset boundary of acceptable grazing areas and ventured into a protected habitat. The beef producer shares the alert with a neighbor who is nearby the cattle's remote location. Upon investigating, the neighbor spots an unfamiliar stock trailer and contacts the authorities to report suspected cattle theft.

The Internet of Cows

Embodiments described herein provide a maintenance-free solar-powered device for animal monitoring that can attach to the animal. Although the description below is written in terms of cattle, the disclosed techniques may be used on other animals that inhabit outdoor environments, such as other types of livestock or wildlife. Although at times the term “cow” is used, the use of the term is not intended to limit the use of the apparatus to females, and it may be used equally well on males, although certain functionality may be sex-dependent, such as estrus detection, which is inherently only relevant to females that go into estrus.

Basic design criteria for the apparatus disclosed below stipulated that the device should operate in any terrain, including mountainous environments. The apparatus is designed to collect health and activity information from the animal, identify the animal's location, analyze the collected data, then transmit animal information to a solar-powered base station, which may be a significant distance from the animal. The base station may then transmit the collected information to a remote computing server that provides storage, further analysis, and provisions for software applications providing a graphical interface capable of communicating geospatially enabled alerts and information to the end user.

Similar to the way in which small network-connected devices have become known as Internet of Things (IoT) devices, the disclosed devices allow a beef producer to remotely monitor animals with what we call Internet of Cows (IoC) devices.

There is a pre-existing market for cattle monitoring technology. These devices are typically collar tags or ear tags, battery-powered devices that attach to a cow collar or ear, and collect various forms of health and location information. However, these devices do not generate geolocation information with a geolocation sensor, and are unable to communicate collected information over the distances required for remote monitoring of animals that inhabit outdoor environments, such as pastures, rangelands and forests. There are other devices that can be attached to cow collars that have been used for creating virtual electric fences, and devices that can be attached to cow collars that generate geolocation information with a geolocation sensor but do not collect health information. The system disclosed below is a novel system that combines geolocation capability with health monitoring and communication of collected information over distances making it feasible for use in outdoor environments such as pastures, rangelands and forests.

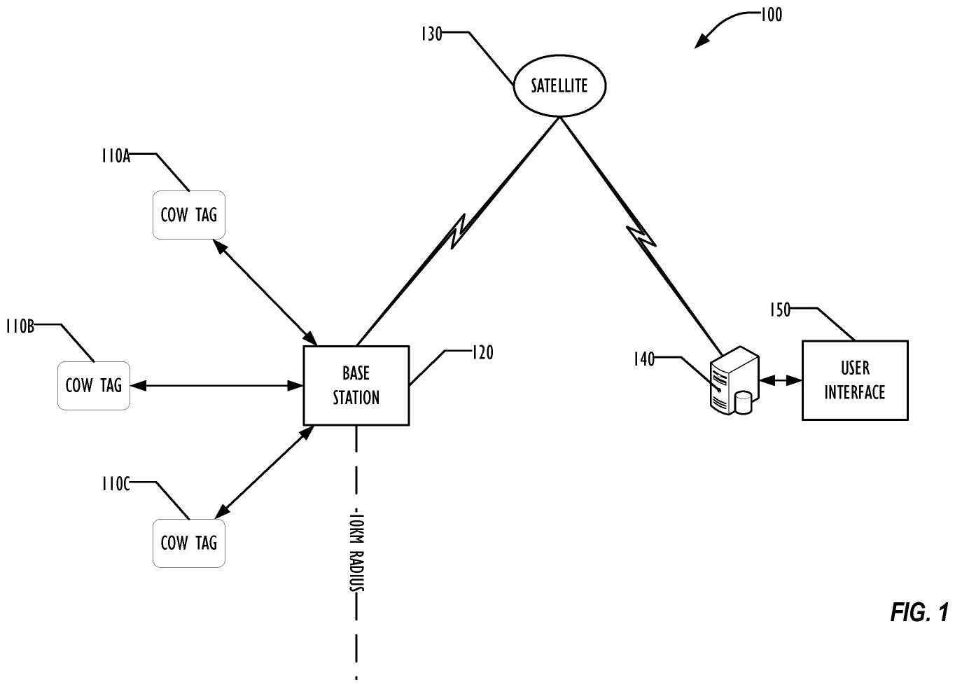

FIG. 1 is a high-level overview illustrating a system for cattle monitoring according to one embodiment. As illustrated in FIG. 1, cow tags 110A, 110B, and 110C communicate with a base station 120. The cow tags are attached to a cow and collect animal health, activity, and geolocation information, and analyze that information. Although the tags described below are described and illustrated in the Figures in terms of ear tags, other embodiments may be implemented as collar or harness tags.

The cow tags 110 can communicate with the base station 120 over a wide area. In one embodiment the cow tags 110 may be anywhere in a radius of approximately 10 km from the base station 120. The base station 120 then communicates with a remote computing server 140 using a satellite 130 and satellite-based Internet. The remote computing server 140 may store, aggregate and analyze the collected data and may support software applications that communicate geospatially enabled animal information to an end user of the graphical user interface 150 to view information about the cattle corresponding to the cow tags 110. Although three cow tags 110 are illustrated in FIG. 1 for clarity of the drawing, any number of animals monitored with the cow tags 110, including thousands of animals. The maximum range between the cow tags 110 and the base station 120 may vary depending upon the operating environment and the particular communication technique and equipment deployed, but in general the system 100 can communicate between cow tags 110 and base station 120 over distances appropriate for cattle in outdoor grazing environments.

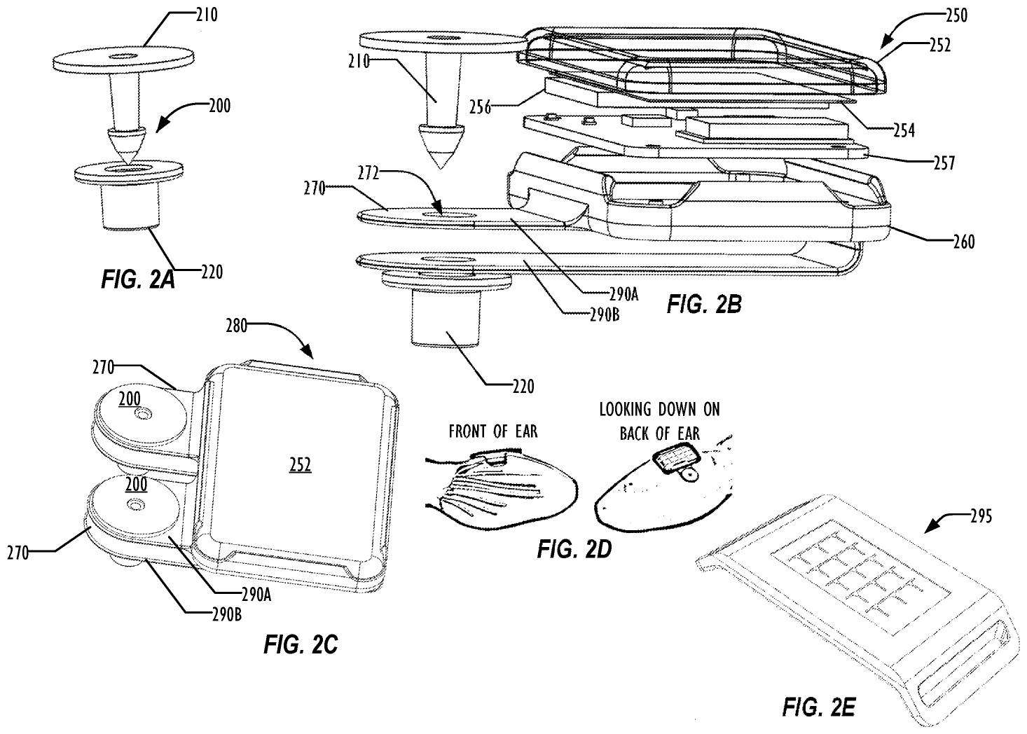

FIGS. 2A, 2B, 2C, and 2D illustrate a mechanism for attaching the cow tags 110 to a cow's ear according to various embodiments. Ear attachment may be accomplished using conventional ear tag installation pliers (not shown) allowing the placement of a commercially available ear tag button 200, in this example comprising a male portion 210 and a female portion 220 through an opening in a mounting tab 270 of ear tag 250. The male portion 210 of the ear tag button 200 snaps into the female portion 220, firmly holding the ear tag 250 in place on the cow's ear. As illustrated in FIG. 2B, a single tab 270 is used for mounting the ear tag 250 to the cow's ear, but other embodiments may use multiple tabs 270 and multiple buttons 200, such as is illustrated in FIG. 2C, which uses two ear tag buttons 200 to attach ear tag 280 onto the cow's ear. Mounting tab 270 may be a tab that is positioned on one side of the cow's ear in one embodiment. However, in other embodiments, such as is illustrated in FIGS. 2B and 2C, mounting tab portions 290A and 290B may be formed for positioning on both sides of the cow's ear, with a gap between them for insertion of the cow's ear. The mounting tab or tabs 270 when engaged with ear tag button or buttons 200 pushed through the cow's ear hold housing 260 onto the cow's ear. Housing 260 may be formed integral with the mounting tab or tabs or as a separate unit to which the mounting tab or tabs may be attached. Housing 260 then holds the electronics for the ear tag 250, 280, described in further detail below. As illustrated in FIG. 2, the electronics may include a circuit board 257, a battery 256, and a solar panel 254. The electronics are disposed in the housing 260, with a cover 252 that attaches to the housing. Preferably, the cover 252 is transparent to allow sunlight to reach the solar panel 254. The housing 260 and cover 252, which may snap into the housing 260, may be made of any desired material, typically a plastic. In some embodiments, the housing 260 is smaller than a stack of credit cards.

Although illustrated as a two-piece button 200 in FIGS. 2A, and 2C, embodiments may use a single piece button with a male portion that extends through the cow's ear and engages with a female portion formed in the mounting tab 270. Other techniques for attaching the cow tags 110 to the cow's ear may be used if desired. For example, FIG. 2E is a perspective view of an embodiment of a tag 295 implemented for use with a collar or harness.

As illustrated in FIG. 2D, the cow tag 110 is attached to the cow's ear so that the solar panel 254 is on the back of the cow's ear, allowing sunlight to reach the solar panel 254.

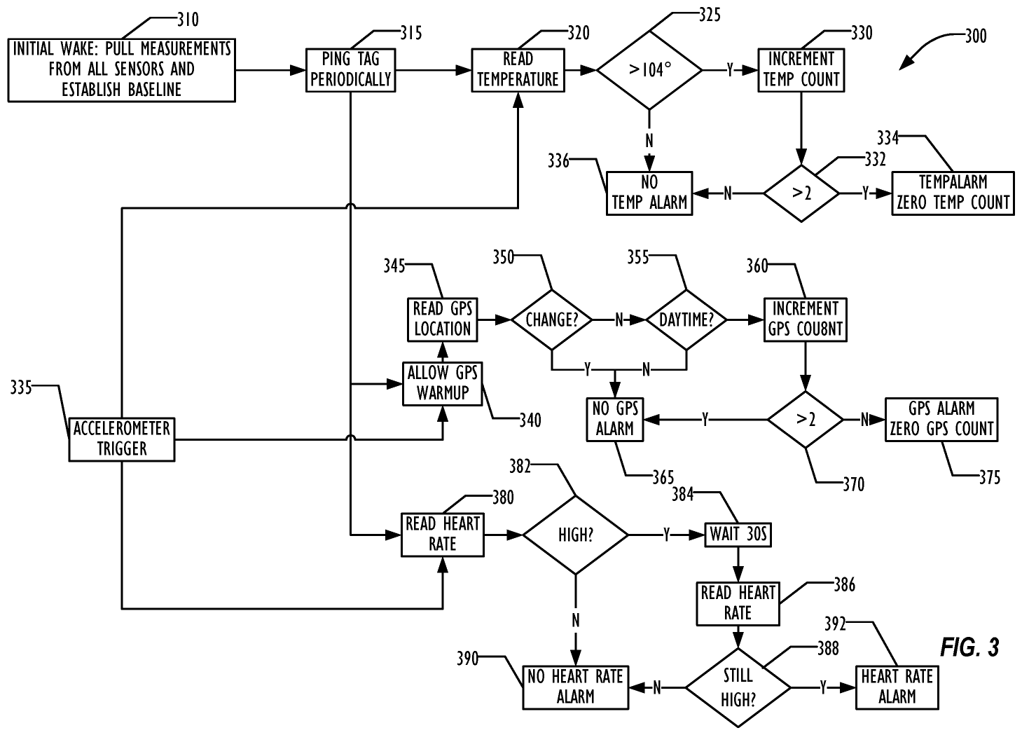

FIG. 3 is a flowchart illustrating an overview of the operation of the cow tags 110 according to one embodiment. In this embodiment, the cow tags 110 contain one or more accelerometers to detect movement, a geolocation sensor, a temperature sensor, and a heart rate sensor. In block 310, when the cow tag 110 initially starts, it pulls measurements from all sensors to establish a baseline. In block 315, the sensors are pinged from time to time. Block 315 may occur on a regular period, e.g. every 30 seconds to ensure that the sensors are read even though no movement is detected by the accelerometer. Alternately, in block 335, the accelerometer may detect movement and trigger reading the sensors outside of the regular period check of block 315.

In this embodiment, three sensors are checked whenever the regular period occurs, or the accelerometer is triggered. In block 320 the temperature sensor is read. If the temperature is above a threshold temperature value, that may be an indication of a problem with the cow. In one embodiment, the threshold is set to a temperature of 104° F. (40° C.). To try to avoid false alarms, a temperature counter may be incremented the first time the temperature is measured as high, and the temperature alarm is only triggered in block 334 if more than two sensor readings in a row as checked in block 332 indicate a high temperature, zeroing the counter when the temperature alarm is triggered. Otherwise, in block 336 no temperature alarm is triggered. Other embodiments may use different techniques for limiting false temperature alarms, such as requiring a different number of consecutive readings to trigger the temperature alarm. Other embodiments may always trigger the temperature alarm any time the temperature exceeds the threshold temperature.

In block 340, the geolocation subsystem is triggered. The geolocation sensor attempts to identify or estimate the real-world geographic location of the cow. Various types of geolocation sensors exist and in one embodiment the geolocation sensor is a unit that receives signals from a satellite geonavigation system, such as the U.S. Global Positioning System (GPS). Other satellite geonavigation systems exist and can be used, such as Russian Global Navigation Satellite System, the Chinese BeiDou Navigation Satellite System, and the European Union's Galileo system, and other satellite-based geonavigation systems are planned by at least Japan and India. Other geolocation techniques that use ground-based radio signals such as real-time kinematics (RTK) and cell towers can be used. Self-contained geolocation techniques such as inertial navigation system (INS) can be used. In FIG. 3, a GPS-based sensor is used, which typically requires a warmup of the GPS sensor in block 340 before a reading can be obtained in block 345. If the location identified in block 345 has not changed as determined in block 350, and the reading is taken during the daytime as determined in block 355, that may be an indication of a problem with the cow. Similar to the temperature false alarm reduction technique, in one embodiment a counter may be used and an alarm signaled in block 375 only if the counter exceeds a threshold value, such as greater than two consecutive readings with no movement. If the cow has moved, or the readings are being taken at night when the cow would normally sleep, no GPS alarm is triggered in block 365.

In block 380, a heart rate sensor reads the heart rate of the cow. If in block 382 the heart rate is above a threshold value (an adult cow has a heart rate of between 48 and 84 beats per minute), as determined in block 382, a heart rate alarm may be triggered. As with the temperature and location techniques, false alarms may be avoided in some embodiments by waiting in block 384 for 30 seconds or any other desired waiting period, then rereading the heart rate in block 386. If the second reading is still above the threshold, as determined in block 388, the heart rate alarm may be triggered in block 392. Otherwise the heart rate alarm will not be triggered in block 390.

Not all embodiments will include all the above-described sensors. Embodiments may use, for example, a combination of accelerometer and geolocation sensors, and omit a heart rate sensor and temperature sensors. Other types of sensors such as a sound sensor (microphone) or inertial measurement unit (IMU) can be used as desired and similar false alarm reduction techniques may be used as desired with those other types of sensors.

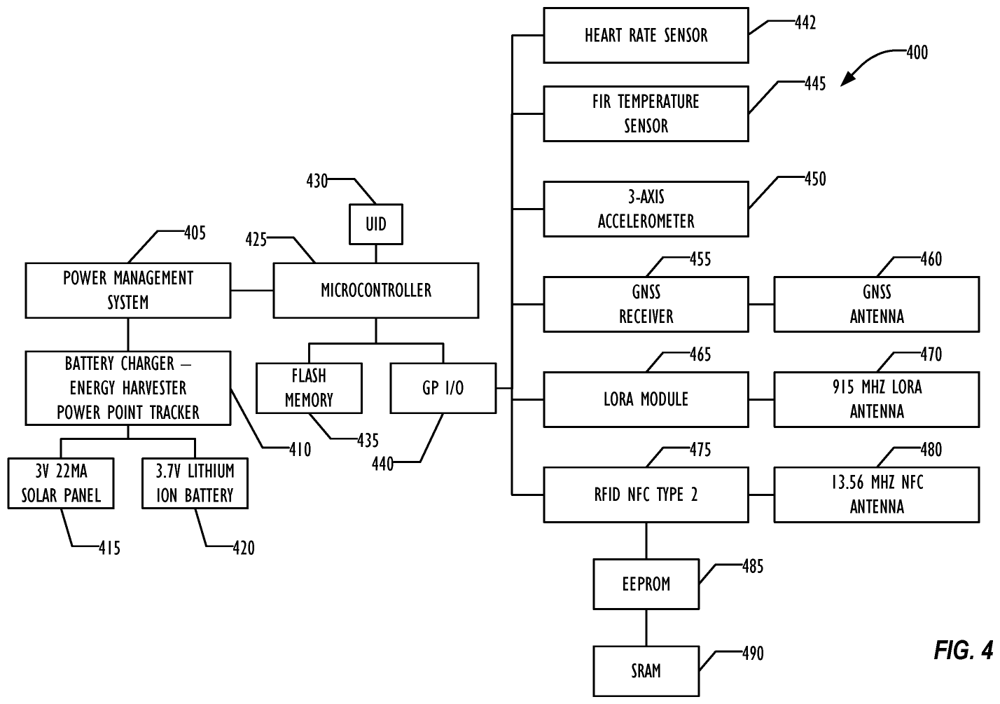

Turning now to FIG. 4, a block diagram illustrates example electronics 400 that may be used to implement a cow tag 110. Preferably, the electronics illustrated in FIG. 4 are disposed with a circuit board that is held within the housing 260. In embodiments that power the cow tag 110 using solar energy, such as the one illustrated in FIG. 4, one side of the housing 260 is formed of a clear material that allows a solar panel disposed with the remainder of the electronics 400 adjacent the clear side of the housing 260, allowing solar energy to interact with the solar panel, causing it to generate electricity.

In this example, a solar panel 415 provides electrical power to a battery charger/energy harvester 410 that then charges a battery such as a lithium ion battery 420. The battery charger/energy harvester 410 boosts the input voltage from the solar panel 415 to the voltage required by the battery 420, maximizing the solar panel output. A power management system unit 405 controls the battery charger/energy harvester 410 to ensure that the battery 420 maintains an appropriate charge level, does not get overcharged, etc. The power management system unit 405 is itself controlled by a microcontroller 425 which may be programmed with software or firmware to operate the cow tag 110 and analyze collected data. A Unique Identifier (UID) unit 430 provides a unique identifier for the cow tag 110, allowing individual cow tags 110 to be addressed remotely. A memory 435, such as a flash memory, may be used for storing collected data and analyzed information along with software, firmware, etc., comprising instructions that when executed cause the microcontroller 425 to perform the actions needed to run the cow tag 110, such as the techniques described in FIG. 3. A general purpose I/O unit communicates between the microcontroller 425 and the various I/O elements such as the sensors and transceivers described below.

In the embodiment illustrated in FIG. 4, a far-infrared temperature sensor 445 is used to detect the temperature of the cow, while a heart rate sensor 442 is used to detect the cow's heart rate. In one embodiment, the heart rate sensor 442 uses optical heart rate monitoring, which is done by sending an IR pulse into the cow's ear and measuring the response. This is done over time and the response varies as a function of the oxygen content in the cow's blood. The oxygen content in the cow's blood fluctuates proportionally to the cow's heart rate. When optical monitoring is used, the heart rate sensor 442 may use a photodiode and a light emitting diode (LED) to do the measurement. In one embodiment, the temperature sensor 445 may measure both ambient temperature as well as the cow's temperature.

A 3-axis accelerometer 450 is used to detect motion of the cow. Motion of the cow may be analyzed to be sure the cow is moving during daytime hours when motion of a healthy cow is to be expected and for more advanced health-monitoring techniques such as monitoring grazing and rumination activity or detecting estrus as based on evidence that a cow in estrus becomes more active.

A geonavigation satellite sensor 455 provides geolocation information to the microcontroller 425, allowing the system to identify the location of the cow. A geonavigation satellite sensor antenna 460 is used to detect signals generated by geonavigation satellites. In some implementations, the geonavigation satellite sensor 455 and the geonavigation satellite sensor antenna 460 may be implemented as a single chip; in others, they may be implemented as separate units.

In one embodiment, communication between the cow tag 110 and the base station 120 uses radio communication that employs Long Range (LoRa) spread spectrum modulation. LoRa is a low power wireless standard intended for providing a cellular style low data rate communications network, using sub-gigahertz radio frequency bands. In North America, a 915 MHz radio band is used, but different bands are used in other areas, such as 433 MHz and 868 MHz in Europe. The LoRa technology covers the physical layer, while other technologies and protocols such as Long-Range Wide Area Network (LoRaWAN) cover the upper networking layers. LoRa enables long-range transmissions with low power consumption with a stated range of 10 km, thus is very useful for something like cow tags on cattle that may wander over large pastures in rural areas. Although the electronic 400 illustrated in FIG. 4 employs LoRa technology, other embodiments may use cellular or other radio technology as desired for the specific use case.

Although shown as two separate units in FIG. 4, in some embodiments the LoRa technology may be implemented as a highly integrated System-in-Package (SiP) module that is integrated with the microcontroller and software stack. In either separate module or SiP embodiments, an antenna 470 is provided for receiving and transmitting radio signals to and from the cow tag 110. As indicated above, in North America, the antenna 470 is designed for 915 MHz signals, but is designed for other frequency bands in other parts of the world. In one embodiment, the antenna 470 is a Planar Inverted F Antenna (PIFA) implemented in microstrip. In other embodiments, a chip antenna, a loop antenna, or other antenna types may be used.

In some embodiments, a Radio Frequency Identification (RFID) Near Field Communication (NFC) Type 2 tag 475 is included with an NFC antenna 480, allowing an end user to access information stored in an individual cow tag 110 when near to the tag. This would typically be done using a smart phone app that supports NFC, but any other type of NFC-capable device could be used. The NFC tag 475 communicates with the microcontroller 425, but may also have access to one or more of an electrically erasable programmable read-only memory (EEPROM) 485 that may be programmed with configuration information and a memory, such as a Static Random-Access Memory (SRAM) 490. The SRAM 490 may hold data collected from the cow before transmittal from the cow tag 110 to the base station 120, for example.

The data collected and locally stored by the cow tag 110 may include acceleration in three axes. In some embodiments, the acceleration uses a sampling frequency of 16 Hz to 32 Hz, based on a 6 to 30 second sampling window. To limit power usage, the accelerometer may activate intermittently, such as every hour, to assess and log the cow's activity. The data collected may include the ambient temperature and the temperature of the cow as measured by the temperature sensor 445, the heart rate as measured by the heart rate sensor 442, or any other data collectable by the cow tag 110. In addition, geolocation information, such as the velocity, heading, and position of the cow may be collected and stored locally. A clock maintained by the microcontroller 425 may provide timestamps for the collected data. The collected data may be locally stored for a fixed period of time, such as 2-7 days, or may be stored until the storage area fills. Older locally stored data may then be deleted as desired.

In some embodiments, the cow tag 110 may permanently store cow identifying information provided by the user that may be used in the analysis of the collected data. This may include one or more of the following or any other cow-specific data that may be considered desirable:

(a) animal owner identifier (such as premises identification number [PIN]);

(b) birthdate;

(c) sex;

(d) breed;

(e) whether the animal is intact;

(f) castration date;

(g) weaning weight and date of weight;

(h) Current weight and date of weight;

(i) Color;

(j) Horned/polled/scurred;

(k) Hip height;

(l) Vaccine record (type of vaccine, date of delivery);

(m) Medication status (type of medication, quantity, date of delivery);

(n) Genetic information;

(o) Offspring (list offspring, breeding partner, date of births);

(p) Breeding history (date of breeding event and breeding partner); and

(q) Last estrus cycle.

In one embodiment the NFC unit 475 may be used to communicate information with the cow tag 110.

The cow tag 110 may implement a storage architecture capable of persistently storing algorithms to process and analyze the accelerometer, geolocation information, and user-provided information (such as birth date, breeding date, sex, etc.). Embodiments may implement a storage architecture that also supports updating, replacing, and adding algorithms, such as over the Internet and over the base station radio to the receiving cow tag. The software for the cow tag 110 may include automatic detection of when to shift to a higher LoRa setting (e.g., close proximity to the base station) using a power-scaled update rate. A boot loader function may allow updating the firmware for the cow tag 110 over the Internet and over the base station radio to the receiving cow tag.

Having described the cow tag 110, we now turn to the base station 120. Once the cow tag 110 has collected data about the cow to which the cow tag 110 is attached, the data needs to reach the beef producer or other user of the data, preferably via the internet. Because beef cattle are typically in remote outdoor locations without existing radio communications coverage (such as WiFi or cellular service), satellite communication is preferable, but direct satellite communication from the cow tag 110 would be difficult, given the infeasibly large battery size required to power direct satellite communications for persistent remote monitoring. Therefore, the cow tags 110 communicate with base station 120 as a central hub that can receive the cow monitoring information and sending it via satellite internet to a remote computing server that the end user would then interface with via computer software. The base station 120 may be placed at a water source such as a stock tank, or other suitable location that is considered somewhat central to the cattle's movements. Most cattle will not stray more than three miles from their water sources. Even if the individual cow moves outside of communication range with the base station, its last known location is recorded, and once the cow returns into communication range, the cow tag 110 automatically begins updating the base station 120 with information collected while out of range.

In some embodiments, the base station 120 is powered by a solar panel and a lithium ion battery due to the rural aspect of where the base station would be located. The data from the individual cow tags would be sent via LoRa communication to the base station and then outputted from the satellite internet capabilities to a satellite, which then communicates with the remote computing server 140 that can be accessed by the user interface 150. There are multiple satellite services that provide satellite internet communication, including ViaSat, Iridium, Globalstar, Starlink and others. The basestation may include additional techniques for geolocation of the individual cow tags, such as an RTK radio modem.

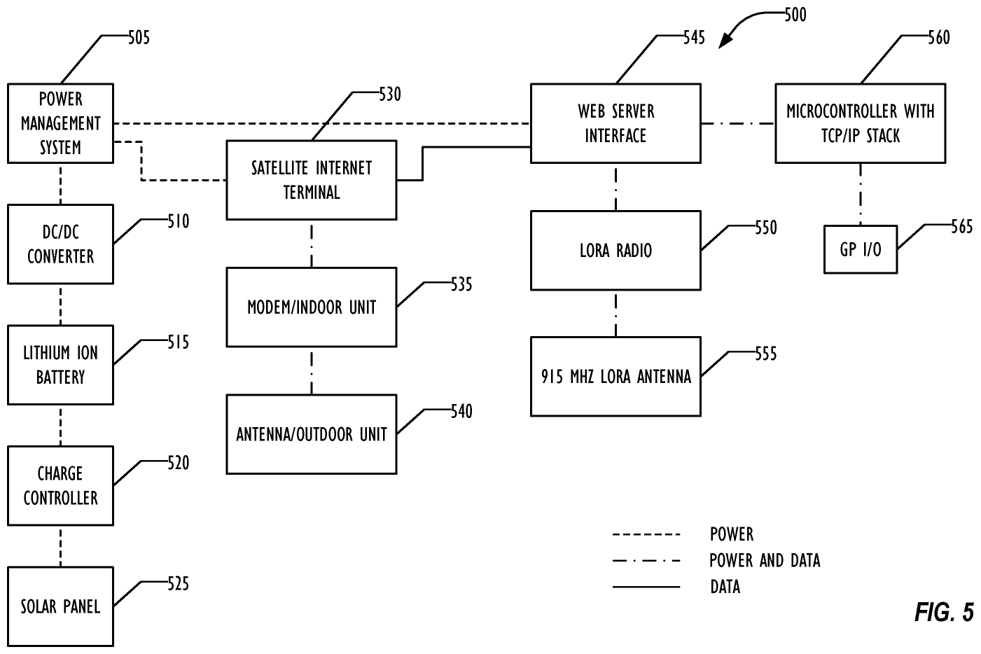

FIG. 5 is a block diagram illustrating a system 500 of components of the base station 120 according to one embodiment. A solar panel 525 provides electrical power to the base station 120. A charge controller 520 converts the output from the solar panel into suitable power for charging a lithium ion battery 515. A DC/DC converter 510 may then, under the control of a power management system 505 convert the battery voltage to the desired voltage and amperage for use by the base station 120. If the base station is positioned in an area where electrical power is available, of course, these components could be omitted and main electrical power could be used with appropriate transformers.

A satellite internet terminal 530, typically provided by the provider of the satellite internet service, is powered by the power management system 505 and provides the communication link between the base station 120 and the satellite 130 used for communication to the remote computing server 140. The satellite internet terminal 530 typically includes a satellite antenna 540, such as a small dish antenna, which is mounted outside of an enclosure, while a modem 535 is typically mounted inside an enclosure with the remainder of the base station for protection against the elements.

A microcontroller 560 that contains a TCP/IP stack controls the non-satellite portion of the base station 120. The microcontroller 560 may be any desired type of microcontroller, such as an INTEL® microprocessor, and ARM® CORTEX® microcontroller, etc. (INTEL is a registered trademark of Intel Corp.; ARM and CORTEX are registered trademarks of ARM Limited.) A general purpose I/O module 565 allows I/O with other devices. A web server software 545 communicates via the satellite internet terminal 530 to the remote computing server 140, via the satellite 130. In embodiments in which the cow tags 110 use LoRa radios, the base station 120 also includes a LoRa radio component 550 and LoRa antenna 555 for communicating with the cow tags 110. The microcontroller is programmed with software executed by the microcontroller 560 that comprises instructions that when executed cause the microcontroller to capture and analyze data from the cow tags 110 and upload it via the web server software 545 through the satellite internet terminal 530 to the remote computing server 140.

In some embodiments, the base station 120 may also comprise NFC, cellular, wireless local network, or other similar components for making a connection between a local device and the base station 120 to allow the local device to communicate with the base station. For example, the general purpose I/O module 565 may allow for a Universal serial bus (USB) connection to the microcontroller 560.

Depending on the size and distribution of pastures within a beef producer's operation, multiple base stations 120 may be used, each communicating via satellite internet to the remote computing server 140.

Although described above as using lithium ion batteries, other types of batteries may be used by the cow tags 110 and base stations 120 as desired.

The remote computing server 140 may comprise any desired computer with Internet capability, and sufficient data storage for storing a desired amount of data collected from the cow tags 110. The remote computing server 140 may be a cloud-based computing server that includes a database, data analytics toolset, and end-user software hosting, for the purposes of aggregating, storing, analyzing, and displaying the collected data and information. Software on the remote computing server 140 provides a user interface, such as a mobile app on a smart phone that can provide a map showing the location and health status of the tagged cattle. Analysis software on the remote computing server 140 may also be used to analyze the collected data on tagged cattle, generating analytic reports and alerts about fertility, disease, calving problems, and unexpected activity or location. For example, a tagged cow that moves outside of a user-prescribed area may have escaped or been stolen. Thus, the system 100 can help beef producers deal with problems that include cattle theft.

In some embodiments, categorization algorithms may run either on the cow tags 110 or in the remote computing server 140 to use the cow tag 110-generated sensor information and user-provided information as needed to detect events of interest and provide alerts in the user interface 150 of those events, such as:

(a) Calving;

(b) Estrus;

(c) Breeding activity;

(d) Outside of user-specified geographic area;

(e) Disease;

(f) Not moving;

(g) Harassment or stress;

Different embodiments may implement the categorization analysis algorithms on the cow tags 110 or the remote computing server 140, based on considerations such as microcontroller computing capabilities, power consumption, on-device storage capacity, integration of historical data, and integration of external data sets. In some embodiments, even if the cow tag 110 has not detected any event that would raise an alarm or be an event of significance, the cow tag 110 may communicate via the LoRa or other radio to the base station to provide a timestamp and location data for the cow. If an event of significance occurs, the cow tag may communicate the event information, including time stamp and location data within a short period of time, such as 1-10 minutes. In some embodiments, when an event of significance has occurred, the cow tag 110 may communicate on a regular basis, such as ½ to 1 hour after the event occurs until an alert associated with the event is cleared by the user via the user interface 150. In some embodiments, to reduce excessive event notifications, the cow tag 110 may send only one of any type of event per day unless the event has been cleared by the user via the user interface 150. Software on the cow tag 110 may look for event clearing commands from the user interface 150 on a regular basis after an event has occurred, such as every ½ to 1 hour, preferably synchronized with geolocation update checks. In some embodiments, an event may be automatically cleared after a predetermined time, e.g., 12 to 24 hours.

The use of the system 100 can enable beef producers to maximize cattle productivity and reduce losses by using the solar-powered, geolocating cow tags 110 and the companion user interface, such as in a mobile app that provides alerts, notifications, and herd maps. The combination of geolocation and health monitoring allows beef producers to maximize herd fertility and nutrition, sustainably manage grazing, automate recordkeeping, and pinpoint cattle that are sick or distressed.

Should a cow tag 110 be removed from the cow, such as being knocked off during grazing activity in woodlands or brushy country, or be removed from the cow by cattle thieves, the cow tag 110 continues to transmit, which facilitates recovery of the device by the end user.

The following examples pertain to further embodiments.

Example 1 is a solar-powered tag for an animal that primarily inhabits outdoor environments, comprising: a housing, attachable to an animal; a solar panel, disposed within the housing and providing electrical power for the solar-powered tag; an accelerometer, disposed within the housing, that when operable measures movement of the animal; a geolocation sensor, disposed within the housing, that when operable determines a geolocation of the animal; a radio, disposed within the housing, having a stated range of at least 10 km; and a microcontroller, disposed within the housing and powered by the solar panel, connected to the accelerometer, the geolocation sensor, and the radio, and programmed to collect and analyze data received from the accelerometer and geolocation sensor and report the analyzed data and a timestamp information to a base station via the radio.

In Example 2 the subject matter of Example 1 optionally includes further comprising: a temperature sensor, disposed within the housing, that when operable measures a temperature of the animal, wherein the microcontroller is further connected to the temperature sensor and is further programmed to collect and analyze temperature data received from the temperature sensor and report the analyzed temperature data to the base station via the radio.

In Example 3 the subject matter of Example 1 optionally includes further comprising: a heart rate sensor, disposed within the housing, that when operable measures a heart rate of the animal, wherein the microcontroller is further connected to the heart rate sensor and is further programmed to collect and analyze heart rate data received from the heart rate sensor and report the analyzed heart rate data to the base station via the radio.

In Example 4 the subject matter of Example 1 optionally includes further comprising: a memory, connected to the microcontroller, adapted to store the collected and analyzed data and timestamp information while the solar-powered tag is out of range of the base station, wherein the microcontroller is further programmed to report the stored data and timestamp information to the base station via the radio upon coming into range of the base station.

In Example 5 the subject matter of Example 1 optionally includes further comprising: a near field communication transponder, connected to the microcontroller, wherein the near field communication transponder communicates with another device with a near field communication transponder for configuration of the solar-powered tag and for reporting collected data to the another device.

In Example 6 the subject matter of Example 1 optionally includes further comprising: a unique identifier unit, connected to or within the microcontroller, providing a unique identifier data for the tag, wherein the microcontroller further communicates the unique identifier data when communicating with the base station.

Example 7 is a solar-powered base station for use in an outdoor area, comprising: a solar panel for providing electrical power to the solar-powered base station; a satellite internet terminal; a radio having a stated range of at least 10 km; and a microcontroller, powered by the solar panel, programmed to: communicate with a plurality of tags attached to monitored animals in the outdoor area using the radio, to collect analyzed sensor data and timestamps from the tags; communicate with a satellite internet service using the satellite internet terminal; and report the collected analyzed sensor data and timestamps via the satellite internet service to a remote computing server.

In Example 8 the subject matter of Example 7 optionally includes further comprising: a web server software, wherein the microcontroller is programmed to communicate with the satellite internet service through the web server software.

In Example 9 the subject matter of Example 7 optionally includes wherein the base station is positioned at a water source for the monitored animals.

In Example 10 the subject matter of Example 7 optionally includes further comprising: a battery connected to the microcontroller and providing electrical power to the microcontroller; and a battery charger connected to charge the battery and powered by the solar panel.

In Example 11 the subject matter of Example 7 optionally includes where the microcontroller is programmed to communicate with a local device.

Example 12 is a system for monitoring animals in outdoor areas, comprising: a plurality of solar-powered tags, each solar-powered tag comprising: a housing, attachable to an animal; a solar panel, disposed within the housing and providing electrical power for the solar-powered tag; an accelerometer, disposed within the housing, that when operable measures a movement of the animal; a geolocation sensor, disposed within the housing, that when operable determines a geolocation of the animal; a radio, disposed within the housing, having a stated range of at least 10 km; and a microcontroller, disposed within the housing and powered by the solar panel, connected to the accelerometer, the geolocation sensor, and the radio, and programmed to collect and analyze data received from the accelerometer and geolocation sensor and report the analyzed data and a timestamp via the radio; a solar-powered base station, designed for placement in the outdoor area at a water source for the monitored animals, comprising: a solar panel for providing electrical power to the solar-powered base station; a satellite internet terminal; a radio having a stated range of at least 10 km; and a microcontroller, powered by the solar panel, programmed to: communicate with the plurality of solar-powered tags using the radio to collect the analyzed data and the timestamps from the solar-powered tags; and communicate with a satellite internet service using the satellite internet terminal; and report the analyzed data and the timestamps to a remote computing server; and a remote computing server, programmed to communicate with the base station via the satellite internet service, wherein the remote computing server collects, aggregates, stores and analyzes information collected from the plurality of solar-powered tags.

In Example 13 the subject matter of Example 12 optionally includes further comprising a user interface for accessing the information collected by the remote computing server.

In Example 14 the subject matter of Example 12 optionally includes wherein the remote computing server is further programmed to analyze the information collected from the plurality of solar-powered tags, generating analytic reports and alerts about fertility, disease, birthing problems, and unexpected activity or location.

In Example 15 the subject matter of Example 12 optionally includes wherein the solar-powered tag continues to communicate with the base station after removal from the animal.

Example 16 is a method for monitoring health, activity, and location of animals in open, outdoor areas, comprising: attaching a solar-powered tag to a monitored animal; collecting and analyzing sensor data and assigning a timestamp corresponding to the monitored animal by the solar-powered tag; transmitting the analyzed sensor data and the timestamp to a solar-powered base station using a radio having a stated range of at least 10 km; receiving the analyzed sensor data and the timestamp from the monitored animal by the solar-powered base station; transmitting the analyzed sensor data and the timestamp corresponding to the monitored animal via satellite-based internet to a remote computing server; analyzing the sensor information and the timestamp received by the remote computing server; and providing geospatially enabled animal information to an end user via a user interface that communicates with the remote computing server.

In Example 17 the subject matter of Example 16 optionally includes wherein the geospatially enabled animal information comprises alerts and animal location maps.

In Example 18 the subject matter of Example 17 optionally includes wherein the alerts comprise birthing alerts.

In Example 19 the subject matter of Example 17 optionally includes wherein the alerts comprise estrus alerts.

In Example 20 the subject matter of Example 17 optionally includes wherein the alerts indicate the monitored animal is not moving.

While certain exemplary embodiments have been described in detail and shown in the accompanying drawings, it is to be understood that such embodiments are merely illustrative of and not devised without departing from the basic scope thereof, which is determined by the claims that follow.

We claim:

1. A solar-powered tag for an animal that primarily inhabits outdoor environments, comprising:
a housing, attachable to an animal;
a solar panel, disposed within the housing and providing electrical power for the solar-powered tag;
an accelerometer, disposed within the housing, that when operable measures movement of the animal;
a geolocation sensor, disposed within the housing, that when operable determines a geolocation of the animal;
a radio, disposed within the housing, having a stated range of at least 10 km; and
a microcontroller, disposed within the housing and powered by the solar panel, connected to the accelerometer, the geolocation sensor, and the radio, and programmed to collect and analyze data received from the accelerometer and geolocation sensor and report the analyzed data and a timestamp information to a base station via the radio.

2. The solar-powered tag of claim 1, further comprising:
a temperature sensor, disposed within the housing, that when operable measures a temperature of the animal,
wherein the microcontroller is further connected to the temperature sensor and is further programmed to collect and analyze temperature data received from the temperature sensor and report the analyzed temperature data to the base station via the radio.

3. The solar-powered tag of claim 1, further comprising:
a heart rate sensor, disposed within the housing, that when operable measures a heart rate of the animal,
wherein the microcontroller is further connected to the heart rate sensor and is further programmed to collect and analyze heart rate data received from the heart rate sensor and report the analyzed heart rate data to the base station via the radio.

4. The solar-powered tag of claim 1, further comprising:
a memory, connected to the microcontroller, adapted to store the collected and analyzed data and timestamp information while the solar-powered tag is out of range of the base station,
wherein the microcontroller is further programmed to report the stored data and timestamp information to the base station via the radio upon coming into range of the base station.

5. The solar-powered tag of claim 1, further comprising:
a near field communication transponder, connected to the microcontroller,
wherein the near field communication transponder communicates with another device with a near field communication transponder for configuration of the solar-powered tag and for reporting collected data to the another device.

6. The solar-powered tag of claim 1, further comprising:
a unique identifier unit, connected to or within the microcontroller, providing a unique identifier data for the tag,
wherein the microcontroller further communicates the unique identifier data when communicating with the base station.

7. A solar-powered base station for use in an outdoor area, comprising:
a solar panel for providing electrical power to the solar-powered base station;
a satellite internet terminal;
a radio having a stated range of at least 10 km; and
a microcontroller, powered by the solar panel, programmed to:
communicate with a plurality of tags attached to monitored animals in the outdoor area using the radio, to collect analyzed sensor data and timestamps from the tags;
communicate with a satellite internet service using the satellite internet terminal; and
report the collected analyzed sensor data and timestamps via the satellite internet service to a remote computing server.

8. The solar-powered base station of claim 7, further comprising:
a web server software,
wherein the microcontroller is programmed to communicate with the satellite internet service through the web server software.

9. The solar-powered base station of claim 7, wherein the base station is positioned at a water source for the monitored animals.

10. The solar-powered base station of claim 7, further comprising:
a battery connected to the microcontroller and providing electrical power to the microcontroller; and
a battery charger connected to charge the battery and powered by the solar panel.

11. The solar-powered base station of claim 7, where the microcontroller is programmed to communicate with a local device.

12. A system for monitoring animals in outdoor areas, comprising:
a plurality of solar-powered tags, each solar-powered tag comprising:
a housing, attachable to an animal;
a solar panel, disposed within the housing and providing electrical power for the solar-powered tag;
an accelerometer, disposed within the housing, that when operable measures a movement of the animal;
a geolocation sensor, disposed within the housing, that when operable determines a geolocation of the animal;
a radio, disposed within the housing, having a stated range of at least 10 km; and
a microcontroller, disposed within the housing and powered by the solar panel, connected to the accelerometer, the geolocation sensor, and the radio, and programmed to collect and analyze data received from the accelerometer and geolocation sensor and report the analyzed data and a timestamp via the radio;
a solar-powered base station, designed for placement in the outdoor area at a water source for the monitored animals, comprising:
a solar panel for providing electrical power to the solar-powered base station;
a satellite internet terminal;
a radio having a stated range of at least 10 km; and
a microcontroller, powered by the solar panel, programmed to:
communicate with the plurality of solar-powered tags using the radio to collect the analyzed data and the timestamps from the solar-powered tags; and
communicate with a satellite internet service using the satellite internet terminal; and
report the analyzed data and the timestamps to a remote computing server; and
a remote computing server, programmed to communicate with the base station via the satellite internet service, wherein the remote computing server collects, aggregates, stores and analyzes information collected from the plurality of solar-powered tags.

13. The system of claim 12, further comprising a user interface for accessing the information collected by the remote computing server.

14. The system of claim 12, wherein the remote computing server is further programmed to analyze the information collected from the plurality of solar-powered tags, generating analytic reports and alerts about fertility, disease, birthing problems, and unexpected activity or location.

15. The system of claim 12, wherein the solar-powered tag continues to communicate with the base station after removal from the animal.

16. A method for monitoring health, activity, and location of animals in open, outdoor areas, comprising:
attaching a solar-powered tag to a monitored animal;
collecting and analyzing sensor data and assigning a timestamp corresponding to the monitored animal by the solar-powered tag;
transmitting the analyzed sensor data and the timestamp to a solar-powered base station using a radio having a stated range of at least 10 km;
receiving the analyzed sensor data and the timestamp from the monitored animal by the solar-powered base station;
transmitting the analyzed sensor data and the timestamp corresponding to the monitored animal via satellite-based internet to a remote computing server;
analyzing the sensor information and the timestamp received by the remote computing server; and
providing geospatially enabled animal information to an end user via a user interface that communicates with the remote computing server.

17. The method of claim 16, wherein the geospatially enabled animal information comprises alerts and animal location maps.

18. The method of claim 17, wherein the alerts comprise birthing alerts.

19. The method of claim 17, wherein the alerts comprise estrus alerts.

20. The method of claim 17, wherein the alerts indicate the monitored animal is not moving.
